(19) United States
(12) Patent Application Publication
(10) Pub. No.: US 2015/0357579 A1
(43) Pub. Date: Dec. 10, 2015

(54) CARBAZOLE DERIVATIVE AND ORGANIC ELECTROLUMINESCENT DEVICE

(71) Applicant: SAMSUNG DISPLAY CO., LTD., Yongin-City (KR)

(72) Inventors: Hiroaki ITOI, Yokohama (JP); Masatsugu UENO, Yokohama (JP)

(21) Appl. No.: 14/724,150

(22) Filed: May 28, 2015

(30) Foreign Application Priority Data
Jun. 6, 2014 (JP) 2014-117511

(51) Int. Cl. H01L 51/00; C09K 11/06; H01L 51/50; C07D 409/14

(52) U.S. Cl. CPC H01L 51/0072; C07D 409/14; C09K 11/06; H01L 51/0074; H01L 51/5004; C09K 2211/1092; C09K 2211/1029; C09K 2211/1007; H01L 51/5016; H01L 2251/558; H01L 51/5056

(57) ABSTRACT

The carbazole derivative is represented by the following General Formula (1).

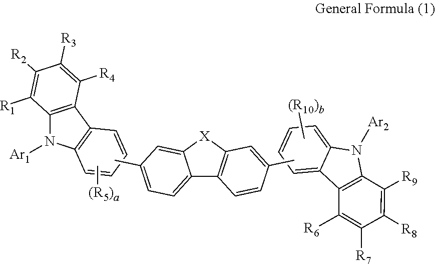

where substitution positions of a dibenzoheterole group at two carbazole rings are the same, and where X, R₁ to R₁₀ Ar₁, Ar₂, a, and b are as defined in the specification.

### CROSS-REFERENCE TO RELATED APPLICATION

Japanese Patent Application No. 2014-117511, filed on Jun. 6, 2014, in the Japanese Patent Office, and entitled: “Carbazole Derivative and Organic Electroluminescent Device,” is incorporated by reference herein in its entirety.

### BACKGROUND

1. Field

Embodiments relate to a carbazole derivative and an organic electroluminescent device.

2. Description of the Related Art

An organic electroluminescent device (organic EL device), which is a so-called self-emitting type device, is being actively developed. The organic EL device is a light-emitting device that emits light from a luminescent material by recombining holes and electrons injected from an anode and a cathode in an emission layer.

A general organic EL device has a laminated structure of an emission layer, a hole transport layer or an electron transport layer for transporting carriers such as holes or electrons to the emission layer, etc.

### SUMMARY

Embodiments are directed to a carbazole derivative represented by the following General Formula (1):

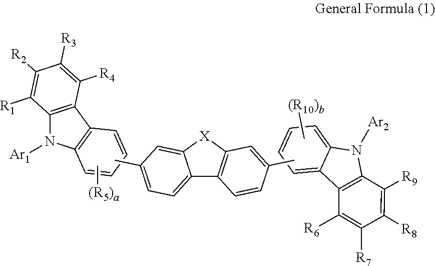

In General Formula (1),

substitution positions of a dibenzoheterole group at two carbazole rings are the same,

X is O, S, SiR₁₁R₁₂ or GeR₁₃R₁₄,

R₁ to R₁₄ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀ aryl group and a substituted or unsubstituted C₁-C₃₀ heteroaryl group, or a substituted or unsubstituted aryl or heteroaryl group formed by fusing at least two adjacent ones of R₁ to R₁₄,

Ar₁ and Ar₂ are independently at least one substituent selected from the group of a substituted or unsubstituted C₁-C₁₅ alkyl group, a substituted or unsubstituted C₆-C₃₀aryl group, and a substituted or unsubstituted C₁-C₃₀ heteroaryl group, and

a and b are independently an integer of 0 to 3.

X may be O, S or SiR₁₁R₁₂.

The substitution positions of the dibenzoheterole ring may be position 3 or position 6 of each carbazole ring.

R₁ to R₁₄, Ar₁ and Ar₂ may be independently a substituent selected from the group of hydrogen, deuterium, a phenyl group and a naphthyl group.

A level of a highest occupied molecular orbital (HOMO) of the carbazole derivative may be from about −5.8 eV to about −5.5 eV.

A difference of energy levels of a triplet excited state (T₁) and a ground state (S₀) of the carbazole derivative may be from about 2.4 eV to about 3.2 eV.

Embodiments are also directed to an organic electroluminescent (EL) device including a carbazole derivative in at least one of a layer between an anode and an emission layer and the emission layer, the carbazole derivative being represented by the following General Formula (1):

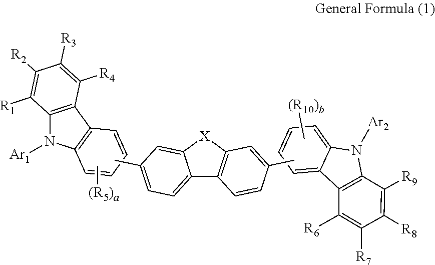

In General Formula (1),

substitution positions of a dibenzoheterole group at two carbazole rings are the same,
X is O, S, SiR₁₁R₁₂ or GeR₁₃R₁₄,

R₁ to R₁₄ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀ aryl group, and a substituted or unsubstituted C₁-C₃₀ heteroaryl group, and a substituted or unsubstituted aryl or heteroaryl group formed by fusing at least two adjacent ones of R₁ to R₁₄,

Ar₁ and Ar₂ are independently at least one selected from the group of a substituted or unsubstituted C₁-C₁₅ alkyl group, a substituted or unsubstituted C₆-C₃₀ aryl group, and a substituted or unsubstituted C₁-C₃₀ heteroaryl group, and

a and b are independently an integer of 0 to 3.

X may be O, S or SiR₁₁R₁₂.

The substitution positions of the dibenzoheterole ring may be position 3 or position 6 of each carbazole ring.

R₁ to R₁₄, Ar₁ and Ar₂ may be independently a substituent selected from the group of hydrogen, deuterium, a phenyl group and a naphthyl group.

A level of a highest occupied molecular orbital (HOMO) of the carbazole derivative may be from about −5.8 eV to about −5.5 eV.

A difference of energy levels of a triplet excited state (T₁) and a ground state (S₀) of the carbazole derivative may be from about 2.4 eV to about 3.2 eV

A thickness of a layer including the carbazole derivative may be from about 3 nm to about 30 nm.

The emission layer may include a fused polycyclic aromatic compound.

The fused polycyclic aromatic compound may include at least one compound selected from the group of an anthracene derivative, a pyrene derivative, a fluoranthene derivative, a chrysene derivative, a benzoanthracene derivative and a triphenylene derivative.

The fused polycyclic aromatic compound may include at least one compound selected from the group of a pyrene derivative and an anthracene derivative represented by the following General Formula (2):

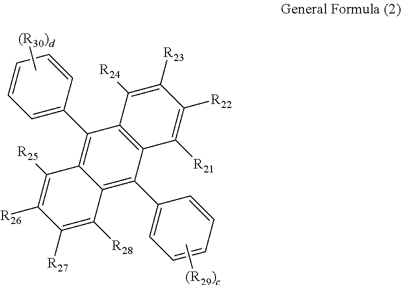

En the above General Formula (2),

R₂₁ to R₃₀ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀aryl group and a substituted or unsubstituted C₁-C₃₀ heteroaryl group, or a substituted or unsubstituted aryl or heteroaryl group formed by fusing at least two adjacent ones of R₂₁ to R₃₀, and

c and d are independently an integer of 0 to 5.

The carbazole derivative may be at least one compounds 1 to 40 (refer to the Detailed Description below).

The fused polycyclic aromatic compound may be at least one of compounds a-1 to a-12 (refer to the Detailed Description below).

### BRIEF DESCRIPTION OF THE DRAWINGS

Features will become apparent to those of skill in the art by describing in detail exemplary embodiments with reference to the attached drawings in which:

FIG. 1 illustrates a schematic diagram depicting an embodiment of an organic EL device according to an embodiment;

FIG. 2 illustrates a schematic diagram depicting an embodiment of an organic EL device manufactured by using a carbazole derivative according to an embodiment; and

FIG. 3 illustrates a schematic diagram depicting another embodiment of an organic EL device manufactured by using a carbazole derivative according to an embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “between” two layers, it can be the only layer between the two layers, or one or more intervening layers may also be present. Like reference numerals refer to like elements throughout.

<1. Carbazole Derivative According to an Embodiment>

First, a carbazole derivative according to an embodiment will be explained. The carbazole derivative according to an embodiment may be used as the host material of an emission layer or as a hole transport material in an organic EL device. The carbazole derivative according to an embodiment may be a compound represented by the following General Formula (1).

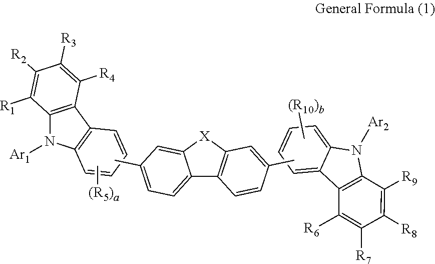

In General Formula (1),

substitution positions of a dibenzoheterole group at two carbazole rings are the same,
X is O, S, SiR₁₁R₁₂ or GeR₁₃R₁₄,

R₁ to R₁₄ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀ aryl group, and a substituted or unsubstituted C₁-C₃₀ heteroaryl group, or a substituted or unsubstituted aryl or heteroaryl group formed by fusing at least two adjacent ones of R₁ to R₁₄,

Ar₁ and Ar₂ are independently at least one substituent selected from the group of a C₁-C₁₅ substituted or unsubstituted alkyl group, a substituted or unsubstituted C₆-C₃₀ aryl group, and a substituted or unsubstituted C₁-C₃₀ heteroaryl group, and

a and b are independently an integer of 0 to 3.

The carbazole derivative represented by the above General Formula (1) has a structure in which two carbazole rings are connected via a dibenzoheterole ring. (The term “dibenzoheterole ring” refers to the central portion of Formula (1) that has a similar structure as a carbazole ring, except having X, as further defined, instead of a nitrogen atom.) The dibenzoheterole ring may have a high electron tolerance. Accordingly, the electron tolerance of the carbazole derivative represented by General Formula (1) may be improved when compared to that of a carbazole derivative not including the dibenzoheterole ring. Thus, the carbazole derivative represented by General Formula (1) may contribute to the improvement of the emission life of the organic EL device.

The carbazole derivative represented by the above General Formula (1) may include a carbazole ring and a dibenzoheterole ring with high planarity. The carbazole derivative represented by General Formula (1) may have high planarity on the whole molecule. The glass transition temperature (Tg), which is an index of heat resistance, may be increased. Thus, the carbazole derivative represented by General Formula (1) may have high heat resistance and may increase the emission life of the organic EL device further. The carbazole derivative represented by General Formula (1) may have high planarity on the whole molecule, and the intermolecular hopping efficiency of holes may be high. High hole transport ability may be attained. The emission efficiency of the organic EL device may be improved by the carbazole derivative represented by General Formula (1).

Position 3 and position 7 of the dibenzoheterole ring may be substituted with the carbazole rings in the carbazole derivative represented by the above General Formula (1). Through the substitution of the carbazole rings at the substitution positions, the carbazole derivative represented by General Formula (1) may restrain radical cleavage due to a nitrogen atom and a heteroatom. The stability of a molecule may be high for the carbazole derivative represented by General Formula (1), and the emission life of the organic EL device may be improved further.

In the carbazole derivative represented by General Formula (1), the substitution positions of the dibenzoheterole at the two carbazole rings may be the same. According to this configuration, the carbazole derivative represented by General Formula (1) may be highly symmetric, and hole mobility may be improved due to intermolecular interaction. Thus, the emission efficiency of the organic EL device using the carbazole derivative represented by General Formula (1) with improved hole mobility may be improved further.

The substitution positions of the dibenzoheterole ring may be, for example, position 3 or position 6 of the carbazole ring in the above General Formula (1). When the dibenzoheterole ring is substituted at position 3 or position 6 of the carbazole ring, the nitrogen atoms of the two carbazole rings may be connected to a conjugated system. The hole mobility of the carbazole derivative represented by General Formula (1) may be improved further. The carbazole derivative in which the dibenzoheterole ring is substituted at position 3 or position 6 of the carbazole rings may decrease the driving voltage of the organic EL device and improve the emission efficiency further.

The number of the substitution position of the dibenzoheterole ring may be designated from the skeleton atom of the ring positioned at the rightmost (excluding carbon at fused position) in a clockwise manner one by one when the dibenzoheterole ring is placed so that a heteroatom is positioned at the bottom part thereof. The number of the substitution position of the carbazole ring may be designated from the skeleton atom of the ring positioned at the rightmost (excluding carbon at fused position) in a counterclockwise manner one by one when the carbazole ring is placed so that a nitrogen atom is positioned at the top part thereof.

In General Formula (1), as an example, X may be O, S or SiR₁₁R₁₂. When X is GeR₁₃R₁₄, two carbazole rings may be connected via a dibenzogermole ring. The stability of the dibenzogermole is lower than other dibenzoheterole rings. Thus, when X is GeR₁₃R₁₄ in the carbazole derivative in General Formula (1), the improvement of the emission life of the organic EL device may not be as great. Accordingly. X may be O, S or SiR₁₁R₁₂ in General Formula (1) in consideration of the increase of the emission life of the organic EL device.

R₁ to R₁₄, Ar₁ and Ar₂ in General Formula (1) may be independently a substituent. The carbazole derivative represented by General Formula (1) may be composed of two carbazole rings and one dibenzoheterole ring, and may have a large molecular weight. R₁ to R₁₄, Ar₁ and Ar₂ in General Formula (1) may be substituted with a substituent having a relatively small molecular weight to suppress an excessive increase of the molecular weight of the carbazole derivative. For example, R₁ to R₁₄, Ar₁ and Ar₂ may be independently substituted with a substituent having a relatively small molecular weight, such as a substituent selected from the group of hydrogen, deuterium, a phenyl group, and a naphthyl group.

The structures of Compounds 1 to 40 are illustrated as particular examples of the carbazole derivative according to an embodiment.

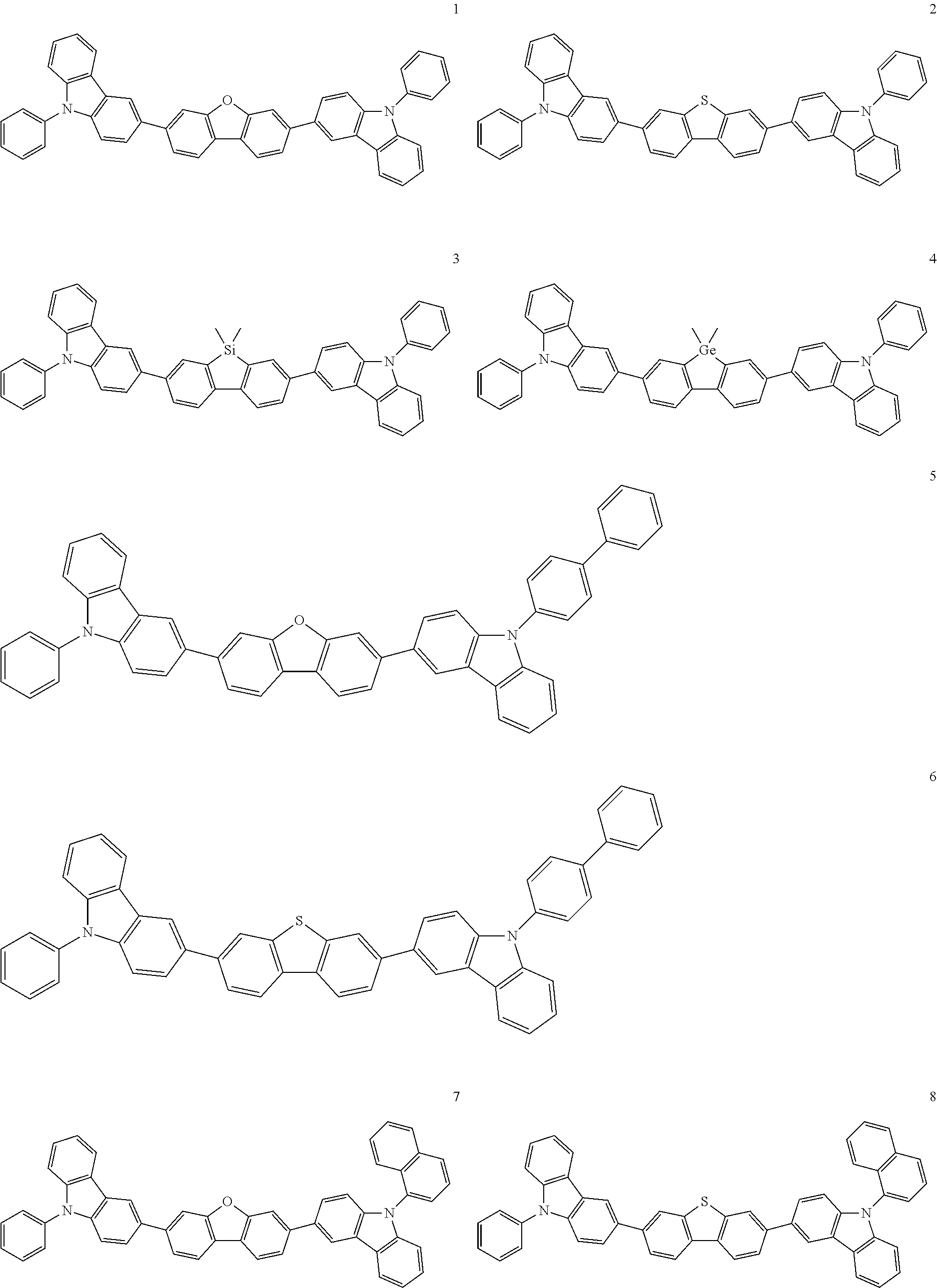

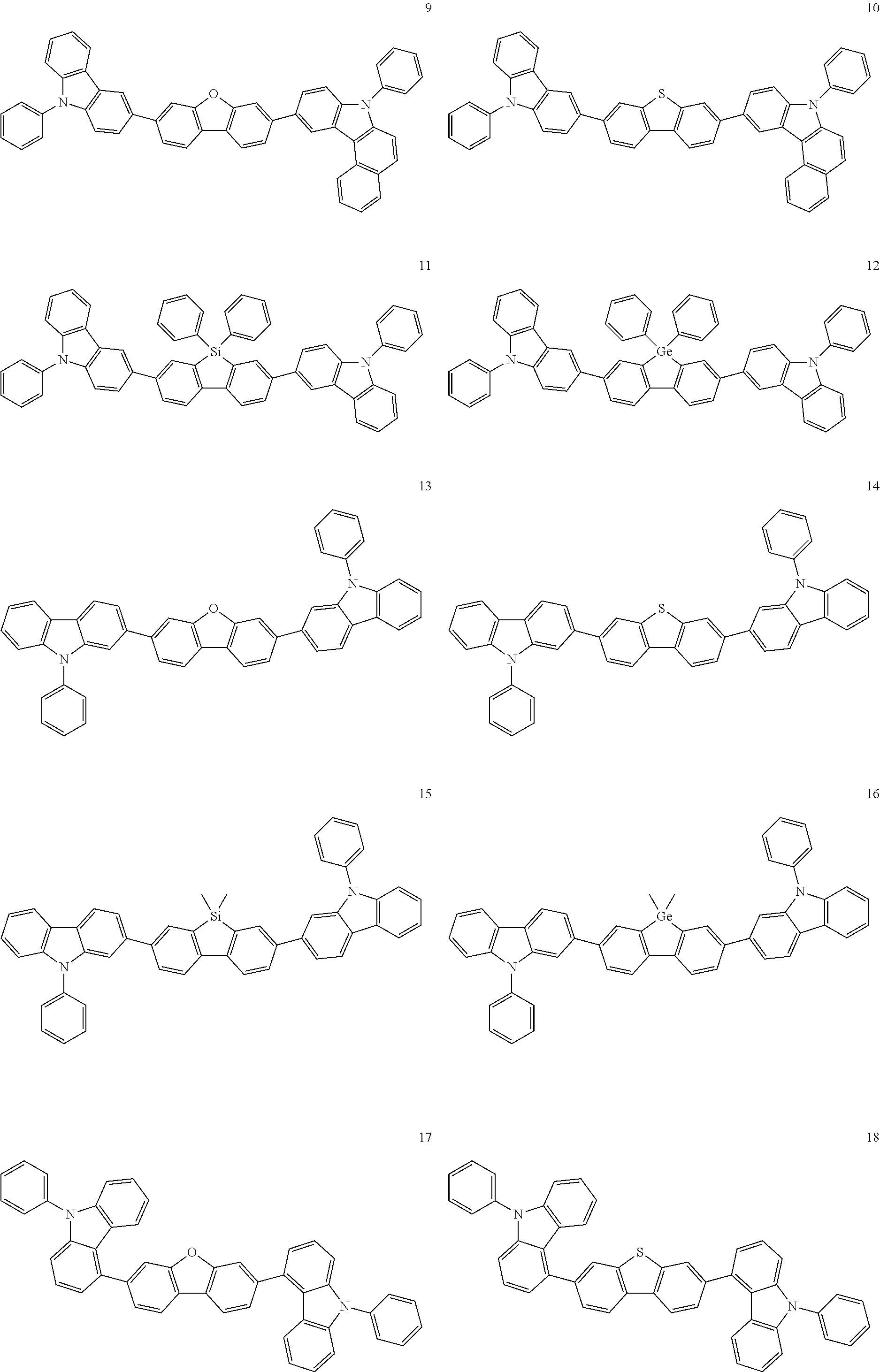

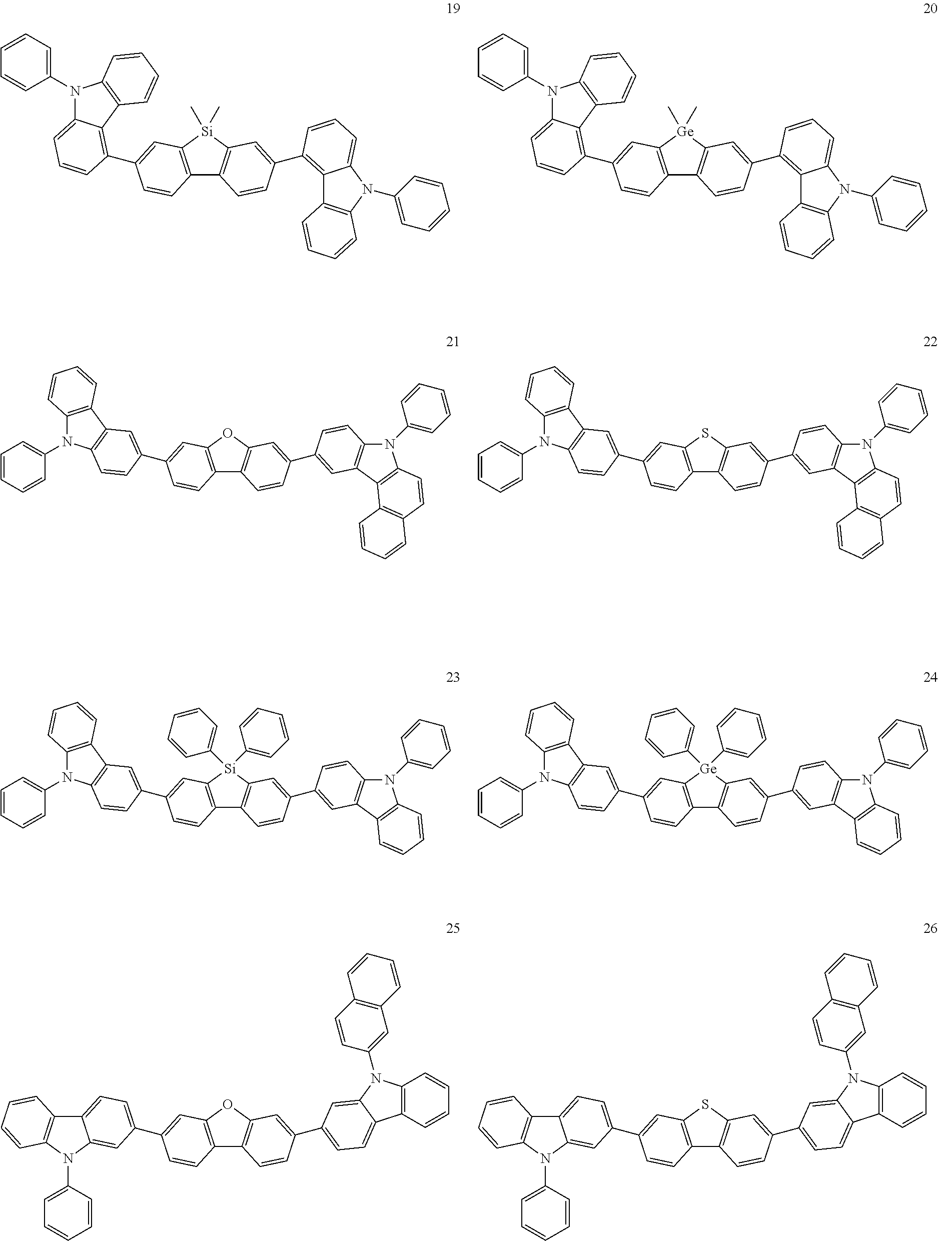

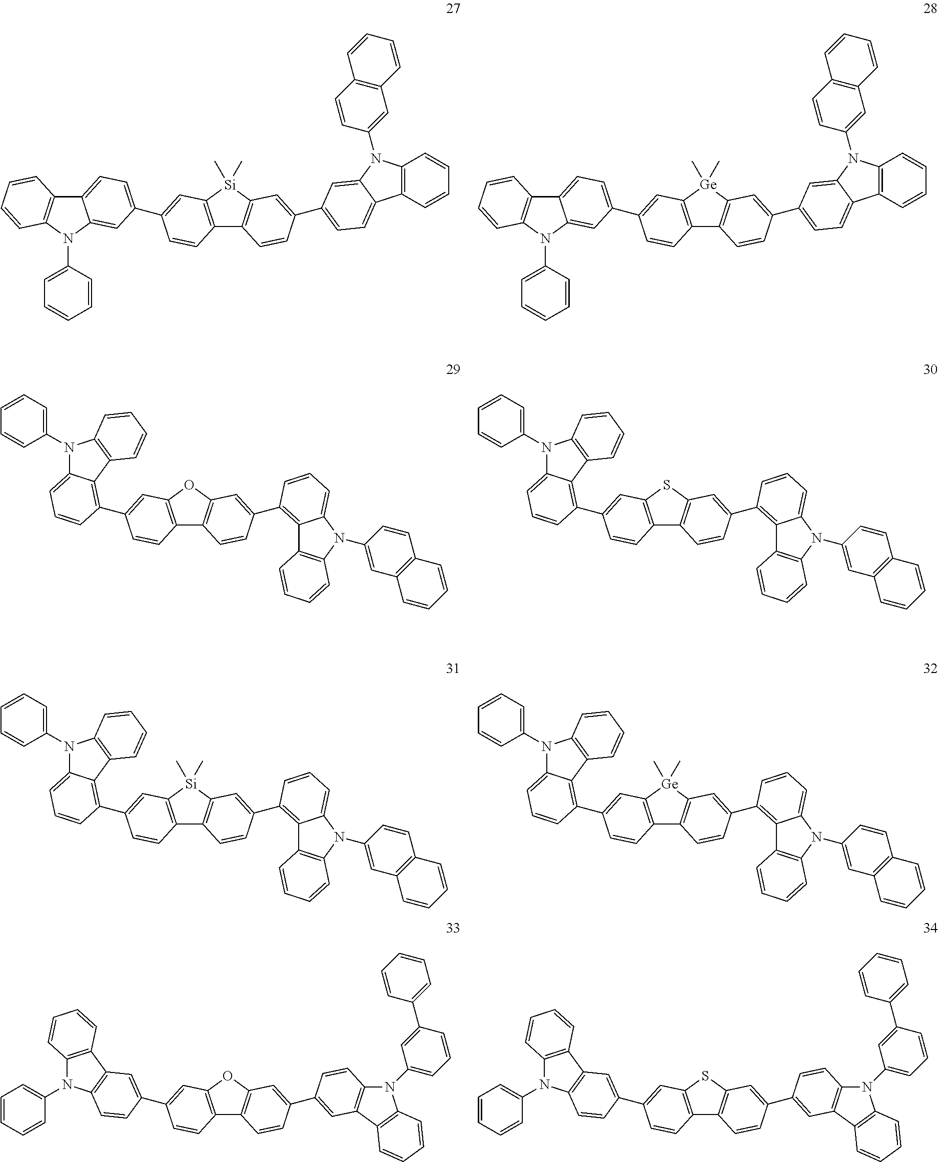

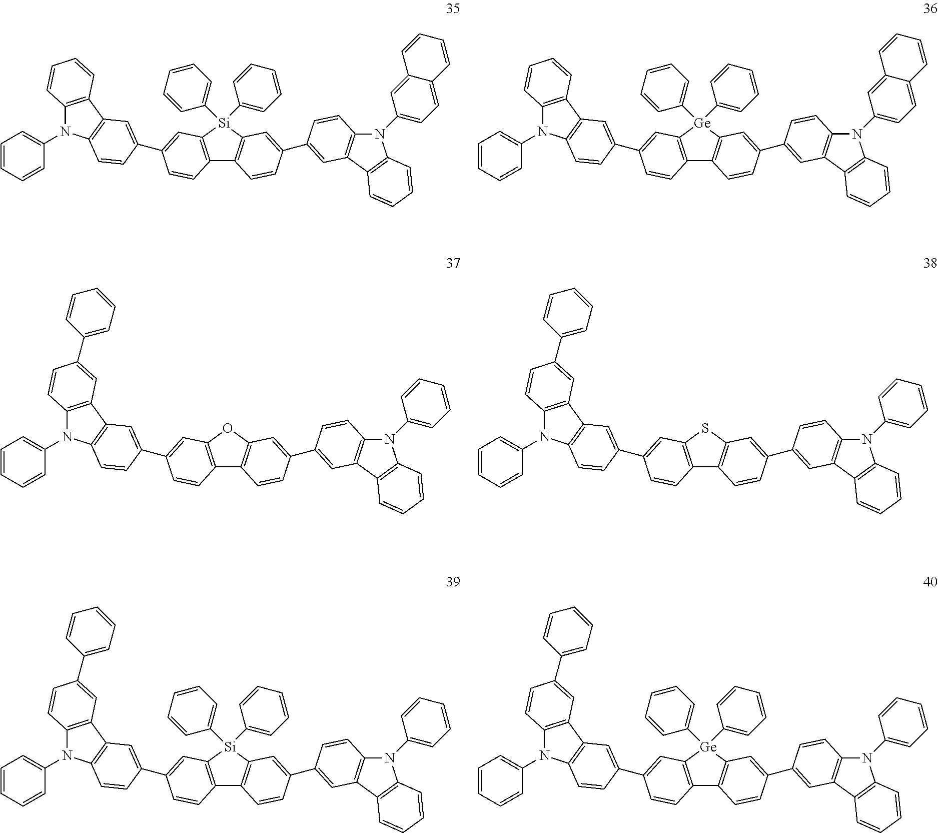

The carbazole derivative according to an embodiment may be used as a host material in an emission layer. In the case that the carbazole derivative according to an embodiment is used as the host material in an emission layer, the driving voltage of the organic EL device may be decreased, and the emission efficiency and the emission life thereof may be improved.

When the carbazole derivative according to an embodiment is used as the host material in an emission layer, the difference between the energy levels of the triplet excited state (T₁) and the ground state (S₀) of the carbazole derivative may be, for example, from about 2.4 eV to about 3.2 eV. When the triplet excited state (T₁) has an energy level in the above range, the carbazole derivative according to an embodiment may efficiently transfer the excited energy with respect to a phosphorescent dopant. For example, the carbazole derivative according to an embodiment may be used as a host material with respect to a green phosphorescent dopant, thereby improving the emission efficiency of the organic EL device.

In addition, the carbazole derivative according to an embodiment may be included in, for example, at least one layer positioned between an emission layer and an anode of an organic EL device (for example, in a hole transport layer) and may be appropriately used as a hole transport material. For example, the carbazole derivative according to an embodiment may be included in a hole injection layer or a hole transport layer as a hole transport material. When the carbazole derivative according to an embodiment is used as the hole transport material, the driving voltage of the organic EL device may be decreased, and the emission efficiency and the emission life thereof may be improved.

When the carbazole derivative according to an embodiment is used as the hole transport material, the HOMO level of the carbazole derivative may be, for example, from about −5.8 eV to about −5.5 eV. For example, by introducing the dibenzoheterole ring between two carbazole rings in the carbazole derivative according to an embodiment, a suitable HOMO level as the hole transport material may be realized. For example, when the HOMO level of the carbazole derivative according to an embodiment is within the above-described range, a driving voltage may be decreased, and emission efficiency may be improved further for an organic EL device using the carbazole derivative as the hole transport material.

The carbazole derivative according to an embodiment may efficiently transport holes with respect to an emission layer by having the HOMO level in the above-described range. For example, the carbazole derivative according to an embodiment may be used as the hole transport material in a blue emitting or green emitting organic EL device. In this case, the driving voltage of the organic EL device may be decreased, and the emission efficiency thereof may be improved.

In addition, when the carbazole derivative according to an embodiment is used as the hole transport material, the thickness of a layer including the carbazole derivative may be, for example, from about 3 nm to about 30 nm. When the thickness of the layer including the carbazole derivative according to an embodiment is more than about 3 nm, hole transporting ability of the carbazole derivative according to an embodiment may be sufficiently exhibited. When the thickness of the layer including the carbazole derivative according to an embodiment is less than about 30 nm, the layer thickness of the whole organic EL device may be suitable, and an undesirable increase in a driving voltage may be avoided.

When the carbazole derivative according to an embodiment is used as the hole transport material, the emission layer may include a fused polycyclic aromatic compound. For example, the emission layer may include at least one fused polycyclic aromatic compound selected from the group of an anthracene derivative, a pyrene derivative, a fluoranthene derivative, a chrysene derivative, a benzoanthracene derivative and a triphenylene derivative. For example, the emission layer may include at least one fused polycyclic aromatic compound selected from the group of the pyrene derivative and the anthracene derivative represented by the following General Formula (2). The fused polycyclic aromatic compound may be included in the emission layer as a host material or as a dopant material.

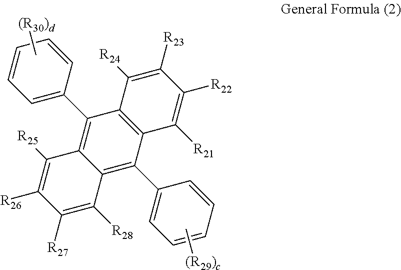

In the above General Formula (2),

R₂₁ to R₃₀ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀ aryl group, and a substituted or unsubstituted C₁-C₃₀ heteroaryl group, or a substituted or unsubstituted aryl or heteroaryl group formed by fusing at least two adjacent ones of R₂₁ to R₃₀, and

c and d are independently an integer of 0 to 5.

The fused polycyclic aromatic compound may be a compound functioning as, for example, a dopant material or a host material in a blue emitting or green emitting emission layer. As described above, the carbazole derivative according to an embodiment may have a suitable HOMO level in the blue emitting or green emitting emission layer. Thus, the emission layer including the fused polycyclic aromatic compound may efficiently receive holes from the carbazole derivative according to an embodiment. When the emission layer includes the fused polycyclic aromatic compound, the organic EL device according to an embodiment may have a decreased driving voltage and improved emission efficiency.

When the carbazole derivative according to an embodiment is used as the hole transport material, examples of the fused polycyclic aromatic compound included in the emission layer may include the following Compounds a-1 to a-12.

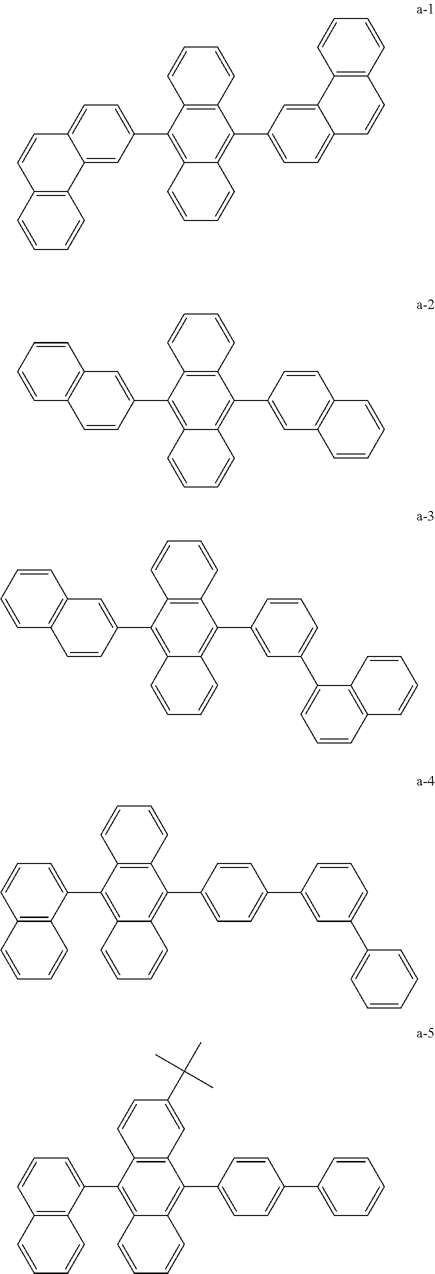

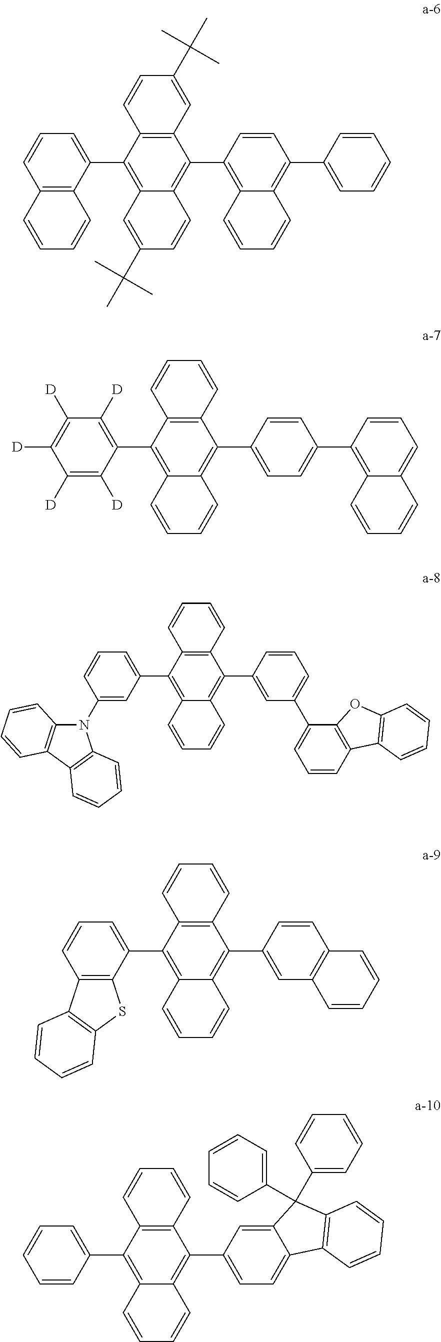

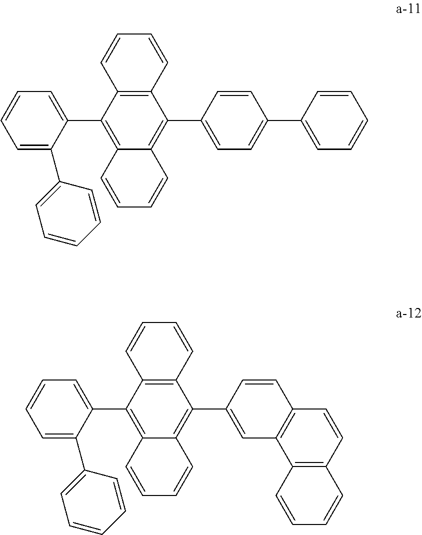

As described above, the carbazole derivative according to an embodiment may have high electron tolerance. When position 3 and position 7 of the dibenzoheterole ring are substituted with carbazole rings, an intramolecular reaction such as radical cleavage, etc. may be restrained. Thus, the life of the organic EL device may be increased by the carbazole derivative according to an embodiment.

The substitution positions of the dibenzoheterole ring at two carbazole rings may be the same in the carbazole derivative according to an embodiment. Thus, the carbazole derivative according to an embodiment may be highly symmetric, may have high hole mobility and may further contribute to the improvement of the emission efficiency of the organic EL device.

Herein, the C₁-C₁₅ alkyl group is a monovalent, linear or branched saturated hydrocarbon or a cyclic saturated hydrocarbon. Examples of the C₁-C₁₅ alkyl group include a methyl group, an ethyl group, a propyl group, an isopropyl group, a cyclopropyl group, a butyl group, an isobutyl group, a tert-butyl group, a cyclobutyl group, a pentyl group, an isopentyl group, a neopentyl group, a cyclopentyl group, a hexyl group, a cyclohexyl group, a heptyl group, an octyl group, a nonyl group, a decyl group, etc.

The C₆-C₃₀ aryl group includes at least one aromatic ring and is a monovalent group having a carbon ring having carbon atoms 6 to 30. Examples of the C₆-C₃₀ aryl group include a phenyl group, a pentalenyl group, an indenyl group, a naphthyl group, an anthracenyl group, an azulenyl group, a heptalenyl group, an acenaphthylenyl group, a phenalenyl group, a fluorenyl group, an anthraquinolyl group, a phenanthryl group, a biphenyl group, a triphenylenyl group, a pyrenyl group, a chrysenyl group, a picenyl group, a perylenyl group, a pentaphenyl group, a pentacenyl group, a tetraphenylene group, a hexaphenyl group, a hexacenyl group, a rubicenyl group, a trinaphthylenyl group, a heptaphenyl group, a pyranthrenyl group, etc.

The C₁-C₃₀ heteroaryl group is a monovalent group having a ring including at least one aromatic ring including at least one heteroatom selected from N, O, P and S and C as the remainder of ring atoms. Examples of the C₁-C₃₀ heteroaryl group include a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolinyl group, an isoquinolinyl group, a benzoimidazolyl group, an imidazopyridinyl group, an imidazopyrimidinyl group, etc.

The above-described C₁-C₁₅ alkyl group, C₆-C₃₀ aryl group, and C₁-C₃₀ heteroaryl group may be substituted with an optional substituent.

<2. Organic EL Device According to an Embodiment>

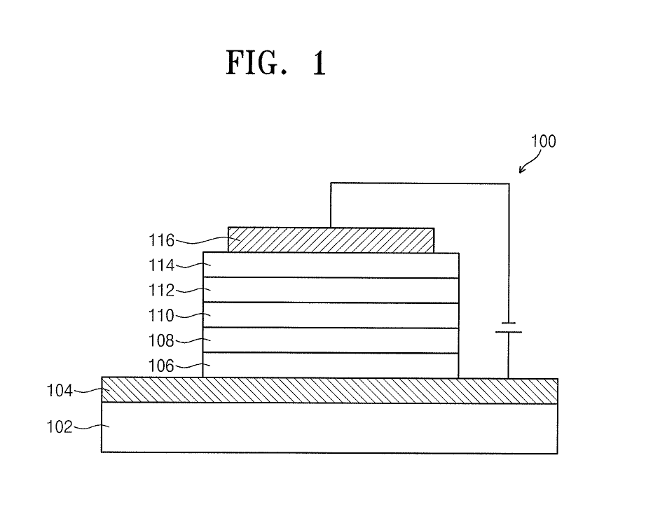

Referring to FIG. 1, an organic EL device according to an embodiment will be explained. FIG. 1 illustrates a schematic diagram illustrating an embodiment of the organic EL device according to an embodiment.

As shown in FIG. 1, an organic EL device 100 according to an embodiment may include a substrate 102, an anode 104 disposed on the substrate 102, a hole injection layer 106 disposed on the anode 104, a hole transport layer 108 disposed on the hole injection layer 106, an emission layer 110 disposed on the hole transport layer 108, an electron transport layer 112 disposed on the emission layer 110, an electron injection layer 114 disposed on the electron transport layer 112 and a cathode 116 disposed on the electron injection layer 114.

The carbazole derivative according to an embodiment may be included in, for example, at least one layer disposed between the anode 104 and the emission layer 110. For example, the carbazole derivative according to an embodiment may be included in at least one of the hole injection layer 106 and the hole transport layer 108.

In addition, the carbazole derivative according to an embodiment may be included as a host material in the emission layer 110.

The structure of the organic EL device 100 may vary from what is shown in FIG. 1. For example, some layers may be omitted in the organic EL device 100 according to an embodiment, or other layers may be added. In some implementations, each layer of the organic EL device 100 may be formed using a plurality of layers.

The substrate 102 may be a support for laminating each layer of the organic EL device 100. The substrate 102 may be a transparent glass substrate, a semiconductor substrate formed using silicon (Si), etc., a flexible resin substrate, etc.

The anode 104 may be disposed on the substrate 102. The anode 104 may be formed using, for example, a metal having high work function, an alloy, a conductive compound, etc. For example, the anode 104 may be formed as a transmissive electrode including indium tin oxide (ITO), indium zinc oxide (IZO), tin oxide (SnO₂), zinc oxide (ZnO), etc. In some implementations, the anode 104 may be formed as a reflective type electrode including magnesium (Mg), aluminum (Al), etc.

The hole injection layer 106 may be disposed on the anode 104. The hole injection layer may enable easy injection of holes from the anode 104. The hole injection layer 106 may be formed by including, for example, the carbazole derivative according to an embodiment. In addition, the hole injection layer 106 may be formed using, for example, 1,4,5,8,9,12-hexaazatriphenylene hexacarbonitrile (HAT(CN)₆), N,N′-diphenyl-N,N′-bis-[4-(phenyl-m-tolyl-amino)-phenyl]-biphenyl-4,4′-diamine (DNTPD), 4,4′,4″-tris(3-methylphenylamino)triphenylamine (m-MTDATA), N,N′-di(1-naphthyl)-N,N′-diphenylbenzidine (NPB), 4,4′,4″-tris{N,N-diamino}triphenylamine (TDATA), 4,4′,4″-tris(N,N-2-naphthylamino)triphenylamine (2-TNATA), polyaniline/dodecylbenzenesulfonic acid (Pani/DBSA), poly(3,4-ethylenedioxythiophene)/poly(4-styrenesulfonate)/(PEDOT/PSS), polyaniline/(camphor)sulfonic acid (Pani/CSA), polyaniline/poly(4-styrenesulfonate (PANI/PSS), etc.

The hole transport layer 108 may be disposed on the hole injection layer 106.

The hole transport layer 108 may provide hole transport from the anode 104 to the emission layer 110. The hole transport layer 108 may be formed by including, for example, the carbazole derivative according to an embodiment. In addition, the hole transport layer 108 may be formed by using, for example, N-phenyl carbazole, polyvinyl carbazole. N,N′-bis(3-methylphenyl)-N,N′-diphenyl-[1,1-biphenyl]-4,4′-diamine (TPD), N,N,N′,N′-tetra-(3-methylphenyl)-3,3′-dimethylbenzidine (HMTPD), carbazolyl)triphenylamine (TCTA), N,N′-di(1-naphthyl)-N,N′-diphenylbenzidine (NPB), etc.

The emission layer 110 may be disposed on the hole transport layer 108. The emission layer 110 may emit light by phosphorescence, fluorescence, etc. The emission layer 110 may be formed using a host material and a dopant material. The dopant material may be one of an emission dopant and a phosphorescent dopant.

As the host material of the emission layer 110, for example, the carbazole derivative according to an embodiment may be used. In addition, the host material of the emission layer 110 may include, for example, tris(8-quinolinolato)aluminum (Alq₃), 4,4′-N,N′-dicarbazole-biphenyl (CBP), poly(n-vinylcarbazole) (PVK), 9,10-di(naphthalene-2-yl)anthracene (ADN), 4,4′,4″-tris(N-carbazolyl)triphenylamine (TCTA), 1,3,5-tris(N-phenylbenzimidazole-2-yl)benzene (TPBI), 3-tert-butyl-9,10-di(naphtho-2-yl)anthracene (TBADN), distyrylarylene (DSA), 4,4′-bis(9-carbazole)-2,2′-dimethyl-biphenyl (dmCBP), etc.

The blue dopant material of the emission layer 110 may include, for example, a styryl derivative such as 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzeneamine (N-BDAVBi), etc., a perylene derivative such as perylene, 2,5,8,11-tetra-t-butylperylene (TBP), etc., a pyrene derivative such as pyrene, 1,1-dipyrene, 1,4-dipyrenylbenzene, 1,4-bis(N,N-diphenylamino)pyrene, etc., and an iridium (Ir) complex compound such as bis[2-(4,6-difluorophenyl)pyridinate](picolinate)iridium(III) (Flrpic), etc. The green dopant material of the emission layer 110 may include, for example, coumarin and the derivative thereof, an iridium complex such as tris(2-phenylpyridin)iridium(III) (Ir(ppy)₃), etc. The red dopant material of the emission layer 110 may include, for example, rubrene and the derivative thereof, 4-dicyanomethylene-2-(p-dimethylaminostyryl)-6-methyl-4H-pyrane (DCM) and the derivative thereof, an iridium complex such as bis(1-phenylisoquinoline)(acetylacetnate)iridium(III) (Ir(piq)₂(acac)), etc., an osmium (Os) complex, a platinum (Pt) complex, etc.

When the carbazole derivative according to an embodiment is included as the host material of the emission layer 110, the dopant material of the emission layer 110 may be, for example, the green phosphorescent dopant.

When the carbazole derivative according to an embodiment is included in at least one of the hole injection layer 106 and the hole transport layer 108, the emission layer 110 may include at least one fused polycyclic aromatic compound selected from the group of an anthracene derivative, a pyrene derivative, a fluoranthene derivative, a chrysene derivative, a benzoanthracene derivative and a triphenylene derivative as the host material or the dopant material. For example, the emission layer 110 may include at least one compound selected from the group of the pyrene derivative and the anthracene derivative represented by the above General Formula (2).

The electron transport layer 112 may be disposed on the emission layer 110. The electron transport layer 112 may enable the transporting of electrons from the cathode 116 to the emission layer 110. The electron transport layer 112 may be formed using, for example, Alq₃ or a material having a nitrogen-containing aromatic ring. The material having a nitrogen-containing aromatic ring may include, for example, a material including a pyridine ring such as 1,3,5-tri[(3-pyridyl)-phen-3-yl]benzene, etc., a material including a triazine ring such as 2,4,6-tris(3′-(pyridin-3-yl)biphenyl-3-yl)-1,3,5-triazine, etc., a material including an imidazole derivative such as 2-(4-(N-phenylbenzoimidazolyl-1-ylphenyl)-9,10-dinaphthylanthracene, etc.

The electron injection layer 114 may be disposed on the electron transport layer 112. The electron injection layer 114 may enable the easy injection of electrons from the cathode 116. The electron injection layer 114 may be formed using, for example, lithium fluoride (LiF), sodium chloride (NaCl), cesium fluoride (CsF), lithium oxide (Li₂O), barium oxide (BaO), etc.

The cathode 116 may be disposed on the electron injection layer 114. The cathode 116 may be formed using, for example, a metal having small work function, an alloy, a conductive compound, etc. For example, the cathode 116 may be formed using, for example, lithium (Li), magnesium (Mg), aluminum (Al), aluminum-lithium (Al—Li), calcium (Ca), magnesium-indium (Mg—In), magnesium-silver (Mg—Ag), etc. In some implementations, the cathode 116 may be formed as a transmissive electrode using ITO, IZO, etc.

Suitable materials for forming each layer of the organic EL device 100 in FIG. 1, in addition to the above-described materials, may be used.

Each layer of the organic EL device 100 according to an embodiment may be formed by selecting an appropriate layer forming method such as a vacuum deposition method, a sputtering method, diverse coating methods. etc. according to the materials used.

For example, an electrode layer such as the anode 104 and the cathode 116 may be formed by a deposition method including an electron beam evaporation method, a hot filament evaporation method, and a vacuum deposition method, a sputtering method, or a plating method such as an electroplating method and an electroless plating method.

The hole injection layer 106, the hole transport layer 108, the emission layer 110 and the electron transport layer 112, the electron injection layer 114, etc. may be formed by, for example, a physical vapor deposition (PVD) method such as a vacuum deposition method, a printing method such as a screen printing method and an ink jet printing method, a laser transcription method and a coating method such as a spin coat method, etc.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### EXAMPLES

Hereinafter, the carbazole derivative and the organic EL device including the carbazole derivative according to an embodiment will be explained in detail referring to examples and comparative examples.

[Synthesis of Carbazole Derivative]

The synthetic method of the carbazole derivative according to an embodiment will be explained in particular illustrating the synthetic methods of Compounds 1 to 5 and 9, as examples.

According to the following Reaction 1, Compound 2 was synthesized as the carbazole derivative according to an embodiment.

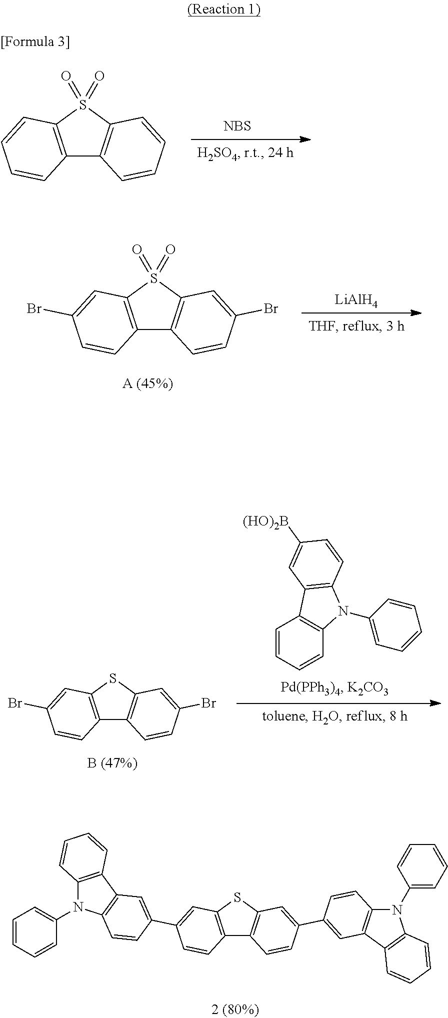

(Synthesis of Compound A)

Under an Ar atmosphere, 2.0 g of dibenzo[b,d]thiophene 5,5-dioxide, 60 mL of concentrated sulfuric acid and 3.29 g of N-bromosuccinimide (NBS) were added to a 500 mL flask, followed by stirring at room temperature for 24 hours. After stirring, the reaction mixture was poured into cold water, precipitated solids were filtered with suction, and solvents were distilled. The crude product thus obtained was washed with water and methanol to produce Compound A as a white solid (1.6 g, yield 45%).

The ¹H-nuclear magnetic resonance (NMR) (DMSO-d₆, 300 MHz) was measured with respect to Compound A, and chemical shift values (δ) of Compound A were 8.33 (d, 2H), 8.11-8.16 (m, 2H), 7.99 (dd, 2H).

(Synthesis of Compound B)

Under an Ar atmosphere, 10.1 g of Compound A and 1.3 g of lithium aluminum hydride (LiAlH₄) were added to a 500 mL flask, followed by heating and refluxing in 174 mL of a tetrahydrofuran (THF) solvent for 3 hours. After cooling in air, the reaction mixture was extracted with ethyl acetate, and magnesium sulfate (MgSO₄) and activated clay were added thereto. After filtering with suction, solvents were distilled. The crude product thus obtained was separated by silica gel column chromatography using a mixture solvent of dichloromethane and hexane to produce Compound B of a pale yellow solid (4.34 g, yield 47%).

Fast atom bombardment mass spectrometry (FAB-MS) was conducted with respect to Compound B, and the measured molecular weight of Compound B was 342.

(Synthesis of Compound 2)

Under an Ar atmosphere, 2.62 g of Compound B, 6.00 g of 9-phenylcarbazole-3-boronic acid, 0.64 g of tetrakis(triphenylphosphine)palladium(0) (Pd(PPh₃)₄) and 1.55 g of potassium carbonate (K₂CO₃) were added to a 300 mL, three-necked flask, followed by heating and stirring in 120 mL of a toluene solvent at 90° C. for 8 hours. After cooling in the air, water was added, an organic layer was separated, and the solvents were distilled. The crude product thus obtained was separated by silica gel column chromatography using a mixture solvent of dichloromethane and hexane and recrystallized using a mixture solvent of toluene/hexane to produce Compound 2 of a white solid (2.40 g, yield 47%).

The molecular weight of Compound 2, as measured by FAB-MS, was 666. With respect to Compound 2, ¹H-NMR (CDCl₃, 300 MHz) was measured, and chemical shift values (δ) of Compound 2 were 8.46 (s, 2H), 8.23 (d, 2H, J=7.82 Hz), 8.04 (d, 2H, J=8.14 Hz), 7.92 (s, 2H), 7.77-7.72 (m, 4H), 7.63-7.61 (m, 8H), 7.52-7.44 (m, 6H), 7.41-7.30 (m, 2H). From the result, the white solid synthesized in Reaction 1 was recognized as Compound 2.

(Synthesis of Compound 1)

The same procedure was conducted as described in Reaction 1 except for using 3,6-dibromodibenzofuran instead of Compound B to produce Compound 1.

(Synthesis of Compound 3)

The same procedure was conducted as described in Reaction 1 except for using 2,7-dibromo-9,9-dimethyl-9H-9-silafluorene instead of Compound B to produce Compound 3.

(Synthesis of Compound 4)

The same procedure was conducted as described in Reaction 1 except for using 2,7-dibromo-9,9-dimethyl-9H-9-dibenzogermole instead of Compound B to produce Compound 4.

(Synthesis of Compound 13)

The same procedure was conducted as described in Reaction 1 except for using 3,6-dibromodibenzofuran instead of dibenzo[b,d]thiophene 5,5-dioxide and using 9-phenylcarbazole-2-boronic acid instead of 9-phenylcarbazole-3-boronic acid to produce Compound 13.

(Synthesis of Compound 17)

The same procedure was conducted as described in Reaction 1 except for using 3,6-dibromodibenzofuran instead of dibenzo[b,d]thiophene 5,5-dioxide and using 9-phenylcarbazole-4-boronic acid instead of 9-phenylcarbazole-3-boronic acid to produce Compound 17.

[Evaluation of HOMO Level of Carbazole Derivative]

The HOMO levels of Compounds 1 to 4, 13 and 17 synthesized above were measured by ultraviolet photoelectron spectroscopy. For the measurement of the HOMO level, an AC-3 photoelectron spectrophotometer by RIKEN KEIKI Co., Ltd. was used. The HOMO levels of Compounds 1 to 4, 13 and 17 thus measured are illustrated in the following Table 1.

TABLE 1

| | HOMO level [eV] |
|---|---|
| Compound 1 | −5.71 |
| Compound 2 | −5.73 |
| Compound 3 | −5.72 |
| Compound 4 | −5.73 |
| Compound 13 | −5.81 |
| Compound 17 | −5.83 |

Referring to Table 1 and the results in Table 2 described below, organic EL devices using Compounds 1 to 4 (Examples 1 to 4) have an even further decreased driving voltage and improved emission efficiency when compared to those of organic EL devices using Compounds 13 and 17 (Examples 5 and 6). Thus, the HOMO level of the carbazole derivative according to an embodiment may be from about −5.8 eV to about −5.5 eV as described above.

[Evaluation of Organic EL Device Including Carbazole Derivative as Hole Transport Material]

An organic EL device 200 including the carbazole derivative according to an embodiment as a hole transport material was manufactured by a vacuum deposition method and the following procedure, and was evaluated.

### Example 1

First, an ITO-glass substrate patterned and washed in advance was surface treated with ozone. The layer thickness of an ITO layer on a glass substrate was about 150 nm. After ozone treatment, a layer was formed using 4,4′,4″-tris(N,N-(2-naphthyl)amino)triphenylamine (2-TNATA, a layer thickness of about 60 nm) as a hole injection material on the ITO layer.

Then, a layer was formed using Compound 1 as a hole transport material to a thickness of about 30 nm to produce a hole transport layer (HTL). In addition, ADN as a host material, doped with 3 wt % of TBP dopant material based on the total amount of the emission layer, was co-deposited to a layer thickness of about 25 nm to produce an emission layer.

Then, a layer was formed using Alq₃ as an electron transport material to a thickness of about 25 nm. A layer was formed using LiF as an electron injection material to a layer thickness of about 1 nm, and a cathode was formed using Al to a layer thickness of about 100 nm to manufacture an organic EL device 200.

### Examples 2 to 6

The same procedure described in Example 1 was conducted except for using Compounds 2 to 4, 13 and 17 instead of Compound 1 to manufacture organic EL devices.

### Comparative Examples 1 to 3

The same procedure described in Example 1 was conducted except for using Compounds 41 to 43 instead of Compound 1 to manufacture organic EL devices. Compound 41 is different from the carbazole derivative according to an embodiment in that three carbazole rings are included, the substitution positions of the carbazole rings at the dibenzoheterole ring are position 2 and position 8. Compounds 42 and 43 are different from the carbazole derivative according to an embodiment in that the dibenzoheterole ring was not included.

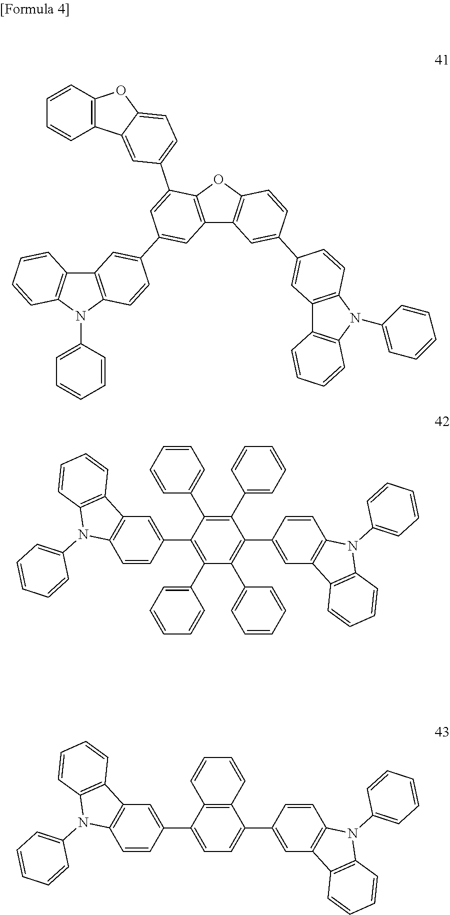 [SMILES: c1ccc(-n2c3ccccc3c3cc(-c4ccc5oc6c(-c7ccc8oc9ccccc9c8c7)cc(-c7ccc8c(c7)c7ccccc7n8-c7ccccc7)cc6c5c4)ccc32)cc1 ; c1ccc(-c2c(-c3ccccc3)c(-c3ccc4c(c3)c3ccccc3n4-c3ccccc3)c(-c3ccccc3)c(-c3ccccc3)c2-c2ccc3c(c2)c2ccccc2n3-c2ccccc2)cc1 ; c1ccc(-n2c3ccccc3c3cc(-c4ccc(-c5ccc6c(c5)c5ccccc5n6-c5ccccc5)c5ccccc45)ccc32)cc1]

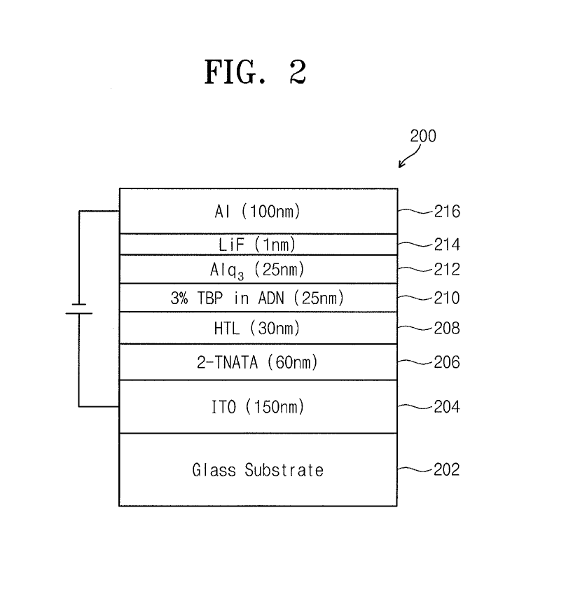

The schematic diagram of the organic EL devices 200 manufactured in Examples 1 to 6 and Comparative Examples 1 to 3 is illustrated in FIG. 2. The organic EL device 200 thus manufactured includes a substrate 202, an anode 204 disposed on the substrate 202, a hole injection layer 206 disposed on the anode 204, a hole transport layer 208 disposed on the hole injection layer 206, an emission layer 210 disposed on the hole transport layer 208, an electron transport layer 212 disposed on the emission layer 210, an electron injection layer 214 disposed on the electron transport layer 212 and a cathode 216 disposed on the electron injection layer 214.

Evaluation results of the organic EL devices 200 of Examples 1 to 6 and Comparative examples 1 to 3 are illustrated in the following Table 2. For the evaluation of the EL properties of the organic EL devices 200, C9920-11 brightness light distribution characteristics measurement system of HAMAMATSU Photonics Co. was used. In the following Table 2, current density was measured at 10 mA/cm², and half life was measured at 1,000 cd/m².

TABLE 2

| | | Current | | | |
|---|---|---|---|---|---|
| | | density | Driving | Emission | Emission |
| | Hole transport | (mA/ | voltage | efficiency | life |
| | material | cm²) | [V] | [cd/A] | LT₅₀ [h] |
| Example 1 | Compound 1 | 10 | 4.7 | 8.4 | 3,500 |
| Example 2 | Compound 2 | 10 | 4.9 | 8.4 | 3,300 |
| Example 3 | Compound 3 | 10 | 5.2 | 7.4 | 2,650 |
| Example 4 | Compound 4 | 10 | 5.7 | 6.9 | 2,400 |
| Example 5 | Compound 5 | 10 | 6.0 | 6.3 | 2,000 |
| Example 6 | Compound 9 | 10 | 6.0 | 6.2 | 2,500 |
| Comparative | Compound 41 | 10 | 6.3 | 7.0 | 1,400 |
| Example 1 | | | | | |
| Comparative | Compound 42 | 10 | 6.5 | 5.2 | 1,000 |
| Example 2 | | | | | |
| Comparative | Compound 43 | 10 | 7.6 | 5.1 | 13,000 |
| Example 3 | | | | | |

Referring to the results in Table 2, the organic EL devices according to Examples 1 to 6 using Compounds 1 to 4, 13 and 17, which are the carbazole derivatives according to embodiments, as the hole transport materials, have increased emission life when compared to those according to Comparative Examples 1 to 3.

For example, the carbazole derivative used in Examples 1 to 6 introduces a dibenzoheterole ring between two carbazole rings and realizes a suitable HOMO level for an organic EL device. Thus, the organic EL devices according to Examples 1 to 6 have decreased driving voltage and improved emission efficiency when compared to those of Comparative Examples 2 and 3. The organic EL devices according to Examples 1 to 6 use the carbazole derivative introducing a dibenzoheterole ring with high electron tolerance. Accordingly, the emission life thereof is found to be increased when compared to those of Comparative Examples 2 and 3.

In the carbazole derivative used in Examples 1 to 6, the substitution positions of the carbazole rings at the dibenzoheterole ring are position 3 and position 7, and radical cleavage due to a nitrogen atom and a heteroatom may be restrained. Thus, the emission life of the organic EL devices according to Examples 1 to 6 may be increased when compared to that of Comparative Example 1, in which the substitution positions of the carbazole rings at the dibenzoheterole ring are different.

In the emission layer of the organic EL devices according to Examples 1 to 6, when the carbazole derivative according to an embodiments is used as the hole transport material, an anthracene derivative (ADN. Compound a-2) may be included, thereby increasing the emission life of the organic EL devices. The thickness of the hole transport layer for Examples 1 to 6 may be about 30 nm, which is found to be a suitable range of a layer including the carbazole derivative according to an embodiment.

Here, in the carbazole derivative used in Examples 1 to 3, X is O, S or SiR₁₁R₁₂ in General Formula (1), and the stability thereof is high. Thus, the emission life of the organic EL devices of Examples 1 to 3 is increased when compared to that of Example 4 in which a carbazole derivative in which X is GeR₁₃R₁₄ and relatively less stability is used.

In the carbazole derivative used in Examples 1 to 4, the dibenzoheterole ring is substituted at position 3 of the carbazole ring, and nitrogen atoms of two carbazole rings may be connected to a conjugated system, thereby improving hole mobility. Therefore, a driving voltage may be decreased further, and emission efficiency may be improved further for the organic EL devices of Examples 1 to 4 when compared to those of Examples 5 and 6 in which a carbazole derivative in which the dibenzoheterole ring is substituted at position 2 or position 4 of the carbazole ring is used.

In addition, as shown in the above Table 1, the organic EL devices of Examples 1 to 4 have an even further decreased driving voltage and even further improved emission efficiency when compared to those of Examples 5 and 6. Thus, a suitable range of the HOMO level of the carbazole derivative according to an embodiment may be from about −5.8 eV to about −5.5 eV.

As described above, the emission life of an organic EL device may be increased by using the carbazole derivative according to an embodiment as a hole transport material.

[Evaluation of Organic EL Device Including Carbazole Derivative as Host Material]

An organic EL device 300 including the carbazole derivative as the host material of an emission layer was manufactured by a vacuum deposition method and the following procedure and was evaluated.

### Example 11

First, an ITO-glass substrate patterned and washed in advance was surface treated with ozone. The layer thickness of an ITO layer on a glass substrate was about 150 nm. After ozone treatment, a layer was formed using 2-TNATA as a hole injection material (a layer thickness of about 60 nm) on the ITO layer.

Then, a layer was formed using HMTPD as a hole transport material to a thickness of about 30 nm. In addition, Compound 1 as a host material doped with 20 wt % of an Ir(ppy)₃ dopant material based on the total amount of the emission layer was co-deposited to a layer thickness of about 25 nm to produce an emission layer.

Then, a layer was formed using Alq₃ as an electron transport material to a thickness of about 25 nm. A layer was formed using LiF as an electron injection material to a layer thickness of about 1 nm, and a cathode was formed using Al to a layer thickness of about 100 nm to manufacture an organic EL device 300.

### Examples 12 to 16

The same procedure described in Example 11 was conducted except for using Compounds 2 to 4, 13 and 17 instead of Compound 1 to manufacture organic EL devices.

### Comparative Examples 11 to 13

The same procedure described in Example 11 was conducted except for using Compounds 41 to 43 instead of Compound 1 to manufacture organic EL devices. Compound 41 is different from the carbazole derivative according to an embodiment in that compound 41 includes three carbazole rings, and the substitution positions of the carbazole rings at the dibenzoheterole ring are position 2 and position 8. Compounds 42 and 43 are different from the carbazole derivative according to an embodiment in that dibenzoheterole ring is not included.

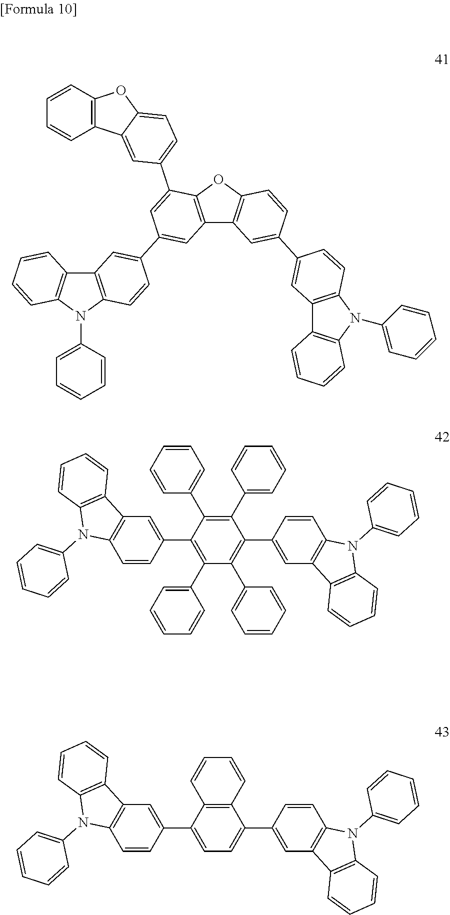 [SMILES: c1ccc(-n2c3ccccc3c3cc(-c4ccc5oc6c(-c7ccc8oc9ccccc9c8c7)cc(-c7ccc8c(c7)c7ccccc7n8-c7ccccc7)cc6c5c4)ccc32)cc1 ; c1ccc(-c2c(-c3ccccc3)c(-c3ccc4c(c3)c3ccccc3n4-c3ccccc3)c(-c3ccccc3)c(-c3ccccc3)c2-c2ccc3c(c2)c2ccccc2n3-c2ccccc2)cc1 ; c1ccc(-n2c3ccccc3c3cc(-c4ccc(-c5ccc6c(c5)c5ccccc5n6-c5ccccc5)c5ccccc45)ccc32)cc1]

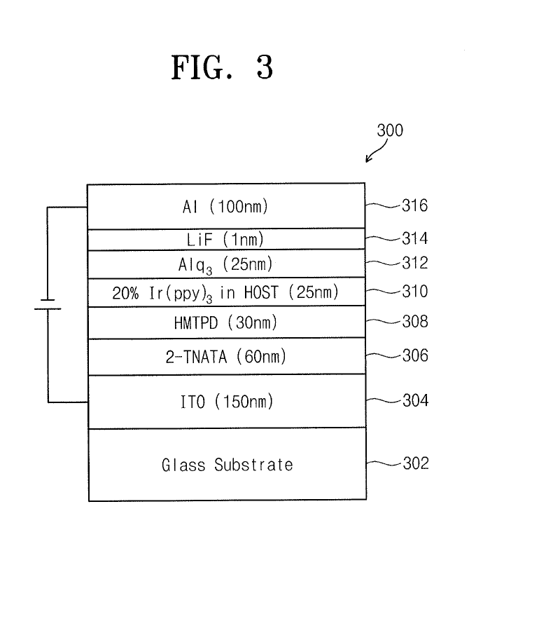

The schematic diagram of the organic EL devices 300 manufactured in Examples 11 to 16 and Comparative Examples 11 to 13 is illustrated in FIG. 3. The organic EL device 300 thus manufactured includes a substrate 302, an anode 304 disposed on the substrate 302, a hole injection layer 306 disposed on the anode 304, a hole transport layer 308 disposed on the hole injection layer 306, an emission layer 310 disposed on the hole transport layer 308, an electron transport layer 312 disposed on the emission layer 310, an electron injection layer 314 disposed on the electron transport layer 312 and a cathode 316 disposed on the electron injection layer 314.

Evaluation results of the organic EL devices 300 of Examples 11 to 16 and Comparative examples 11 to 13 are illustrated in the following Table 3. For the evaluation of the EL properties of the organic EL devices 300, C9920-11 brightness light distribution characteristics measurement system of HAMAMATSU Photonics Co. was used. In the following Table 3, current density was measured at 10 mA/cm², and half life was measured at 1,000 cd/m².

TABLE 3

| | | Current | | | |
|---|---|---|---|---|---|
| | | density | Driving | Emission | Emission |
| | | (mA/ | voltage | efficiency | life |
| | Host material | cm²) | [V] | [cd/A] | LT₅₀ [h] |
| Example 11 | Compound 1 | 10 | 4.0 | 35.7 | 2,600 |
| Example 12 | Compound 2 | 10 | 4.2 | 35.1 | 2,500 |
| Example 13 | Compound 3 | 10 | 4.4 | 33.0 | 2,150 |
| Example 14 | Compound 4 | 10 | 4.8 | 33.1 | 2,000 |
| Example 15 | Compound 13 | 10 | 4.1 | 35.2 | 2,100 |
| Example 16 | Compound 17 | 10 | 4.2 | 36.1 | 2,600 |
| Comparative | Compound 41 | 10 | 5.2 | 30.1 | 1,400 |
| Example 11 | | | | | |
| Comparative | Compound 42 | 10 | 5.3 | 29.3 | 1,000 |
| Example 12 | | | | | |
| Comparative | Compound 43 | 10 | 5.5 | 28.7 | 1,200 |
| Example 13 | | | | | |

Referring to the results in Table 3, the organic EL devices according to Examples 11 to 16 using Compounds 1 to 4, 13 and 17, which are the carbazole derivatives according to embodiments, as the host materials of an emission layer, have decreased driving voltage, increased emission efficiency and increased emission life when compared to those according to Comparative Examples 11 to 13.

For example, the carbazole derivative used in Examples 11 to 16 introduces a dibenzoheterole ring between two carbazole rings and realizes a suitable energy level of the triplet excited state (T₁) for a phosphorescent dopant. Thus, the organic EL devices according to Examples 11 to 16 have decreased driving voltage and improved emission efficiency when compared to those of Comparative Examples 12 and 13. The organic EL devices according to Examples 11 to 16 use the carbazole derivative introducing a dibenzoheterole ring with high electron tolerance. Accordingly, the emission life thereof is found to be increased when compared to those of Comparative Examples 12 and 13.

In the carbazole derivative used in Examples 11 to 16, the substitution positions of the carbazole rings at the dibenzoheterole ring are position 3 and position 7, and radical cleavage due to a nitrogen atom and a heteroatom may be restrained. Thus, the emission life of the organic EL devices according to Examples 11 to 16 may be increased when compared to that of Comparative Example 11, in which the substitution positions of the carbazole rings at the dibenzoheterole ring are different.

Here, in the carbazole derivatives used in Examples 11 to 13, X is O, S or SiR₁₁R₁₂ in General Formula (1), and the stability thereof is high. Thus, the emission life of the organic EL devices of Examples 11 to 13 is increased when compared to that of Example 14 in which a carbazole derivative of which X is GeR₁₃R₁₄ and having relatively less stability was used.

As described above, the use of the carbazole derivative according to an embodiment as the host material of the emission layer also contributes to the increase of the emission life of the organic EL device.

The carbazole derivative according to an embodiment has high electron tolerance and is substituted with the carbazole rings at position 3 and position 7 of the dibenzoheterole ring. Thus, radical cleavage may be restrained, and the long life of the organic EL device may be realized. The substitution positions of the dibenzoheterole ring at the two carbazole rings may be the same. Accordingly, the molecule of the carbazole derivative is highly symmetric and has improved hole mobility, thereby improving the emission efficiency of the organic EL device.

In the above-described embodiments, an organic EL device using the carbazole derivative according to an embodiment as the hole transport material or the host material of the emission layer has been explained. It is to be understood that the carbazole derivative may be used in other luminescent devices or luminescent apparatuses besides the illustrated organic EL devices. For example, the carbazole derivative according to an embodiment may be used in other luminescent devices or luminescent apparatuses. The organic EL devices shown in FIGS. 1 to 3 may be used in an organic EL display of an active-matrix driving type as well as in an organic EL display of a passive-matrix driving type.

By way of summation and review, to improve the emission efficiency and the emission life of the organic EL device, various compounds have been examined as the material of each layer. For example, an aromatic amine compound has been suggested as the material for an organic EL device. A heterocyclic compound substituted with a carbazole ring may be used as a host material of an emission layer. A carbazole derivative substituted with a fused ring may be used as a hole transport material. However, such compounds may have insufficient electron tolerance, and an organic EL device using the compounds may have a defect of short emission life. Thus, a material compound capable of further improving the emission life of the organic EL device is desirable.

Embodiments provide a carbazole derivative having greater stability and that may provide an increased emission life of an organic EL device. Embodiment further provide an organic EL device including the carbazole derivative having a decreased driving voltage and an increased emission life and emission efficiency.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope thereof as set forth in the following claims.

What is claimed is:

1. A carbazole derivative represented by the following General Formula (1): 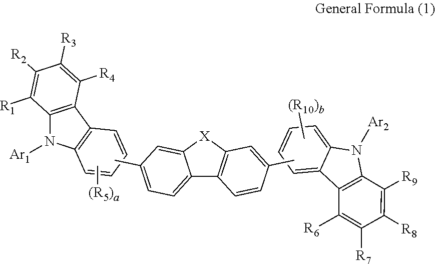
in General Formula (1),
substitution positions of a dibenzoheterole group at two carbazole rings are the same,
X is O, S, SiR₁₁R₁₂ or GeR₁₃R₁₄,
R₁ to R₁₄ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀ aryl group and a substituted or unsubstituted C₁-C₃₀ heteroaryl group, or a substituted or unsubstituted aryl or heteroaryl group formed by fusing at least two adjacent ones of R₁ to R₁₄,
Ar₁ and Ar₂ are independently at least one substituent selected from the group of a substituted or unsubstituted C₁-C₁₅ alkyl group, a substituted or unsubstituted C₆-C₃₀aryl group, and a substituted or unsubstituted C₁-C₃₀ heteroaryl group, and
a and b are independently an integer of 0 to 3.

2. The carbazole derivative as claimed in claim 1, wherein X is O, S or SiR₁₁R₁₂.

3. The carbazole derivative as claimed in claim 1, wherein the substitution positions of the dibenzoheterole ring are position 3 or position 6 of each carbazole ring.

4. The carbazole derivative as claimed in claim 1, wherein R₁ to R₁₄, Ar₁ and Ar₂ are independently a substituent selected from the group of hydrogen, deuterium, a phenyl group and a naphthyl group.

5. The carbazole derivative as claimed in claim 1, wherein a level of a highest occupied molecular orbital (HOMO) of the carbazole derivative is from about −5.8 eV to about −5.5 eV.

6. The carbazole derivative as claimed in claim 1, wherein a difference of energy levels of a triplet excited state (T₁) and a ground state (S₀) of the carbazole derivative is from about 2.4 eV to about 3.2 eV.

7. An organic electroluminescent (EL) device comprising a carbazole derivative in at least one of a layer between an anode and an emission layer and the emission layer,
the carbazole derivative being represented by the following General Formula (1): 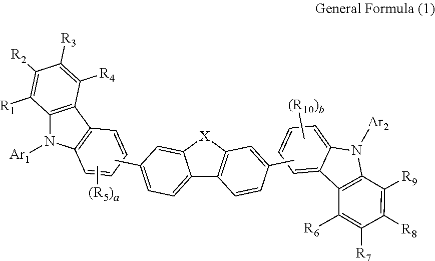
in General Formula (1),
substitution positions of a dibenzoheterole group at two carbazole rings are the same,
X is O, S, SiR₁₁R₁₂ or GeR₁₃R₁₄,
R₁ to R₁₄ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀ aryl group, a substituted or unsubstituted heteroaryl group and a substituted or unsubstituted aryl or heteroaryl group formed by fusing at least two adjacent ones of R₁ to R₁₄,
Ar₁ and Ar₂ are independently at least one selected from the group of a substituted or unsubstituted C₁-C₁₅ alkyl group, a substituted or unsubstituted C₆-C₃₀ aryl group, and a substituted or unsubstituted C₁-C₃₀ heteroaryl group, and
a and b are independently an integer of 0 to 3.

8. The organic EL device as claimed in claim 7, wherein X is O, S or SiR₁₁R₁₂.

9. The organic EL device as claimed in claim 7, wherein the substitution positions of the dibenzoheterole ring are position 3 or position 6 of each carbazole ring.

10. The organic EL device as claimed in claim 7, wherein R₁ to R₁₄, Ar₁ and Ar₂ are independently a substituent selected from the group of hydrogen, deuterium, a phenyl group and a naphthyl group.

11. The organic EL device as claimed in claim 7, wherein a level of a highest occupied molecular orbital (HOMO) of the carbazole derivative is from about −5.8 eV to about −5.5 eV.

12. The organic EL device as claimed in claim 7, wherein difference of energy levels of a triplet excited state (T₁) and a ground state (S₀) of the carbazole derivative is from about 2.4 eV to about 3.2 eV

13. The organic EL device as claimed in claim 7, wherein a thickness of a layer including the carbazole derivative is from about 3 nm to about 30 nm.

14. The organic EL device as claimed in claim 7, wherein the emission layer includes a fused polycyclic aromatic compound.

15. The organic EL device as claimed in claim 14, wherein the fused polycyclic aromatic compound is at least one compound selected from the group of an anthracene derivative, a pyrene derivative, a fluoranthene derivative, a chrysene derivative, a benzoanthracene derivative and a triphenylene derivative.

16. The organic EL device as claimed in claim 14, wherein the fused polycyclic aromatic compound is at least one compound selected from the group of a pyrene derivative and an anthracene derivative represented by the following General Formula (2): 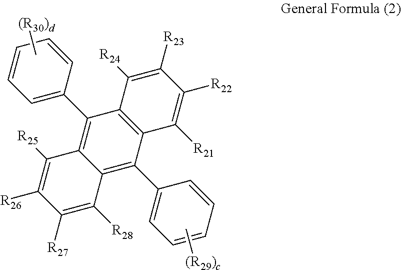
in the above General Formula (2),
R₂₁ to R₃₀ are independently at least one substituent selected from the group of hydrogen, deuterium, halogen, a substituted or unsubstituted silyl group, a substituted or unsubstituted C₁-C₁₅ alkyl group, a substituted or unsubstituted C₆-C₃₀aryl group and a substituted or unsubstituted C₁-C₃₀ heteroaryl group, or a substituted or unsubstituted aryl or heteroaryl group formed by fusing at least two adjacent ones of R₂₁ to R₃₀, and
c and d are independently an integer of 0 to 5.

17. The organic EL device as claimed in claim 7, wherein the carbazole derivative is at least one of the following compounds 1 to 40:

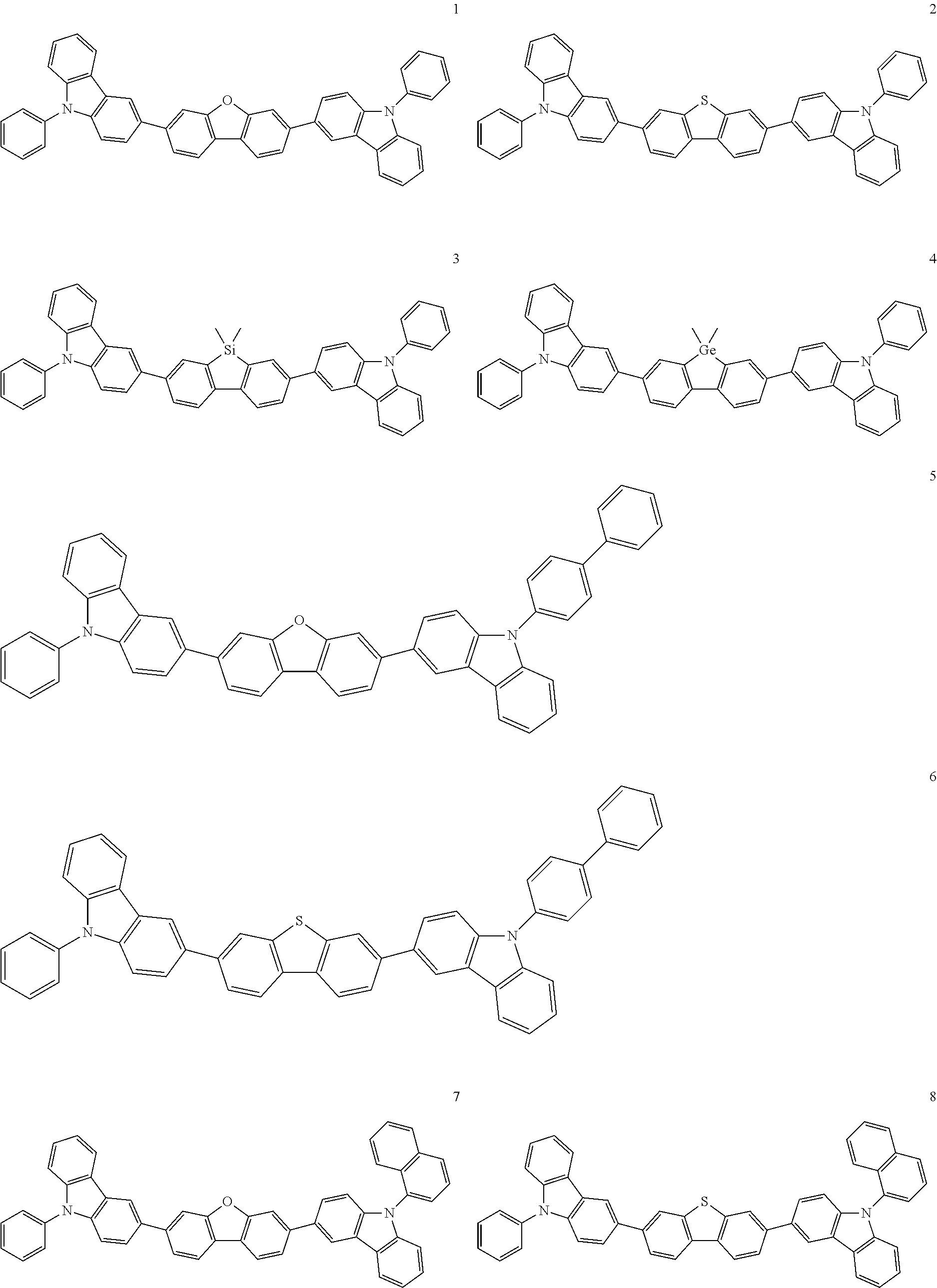

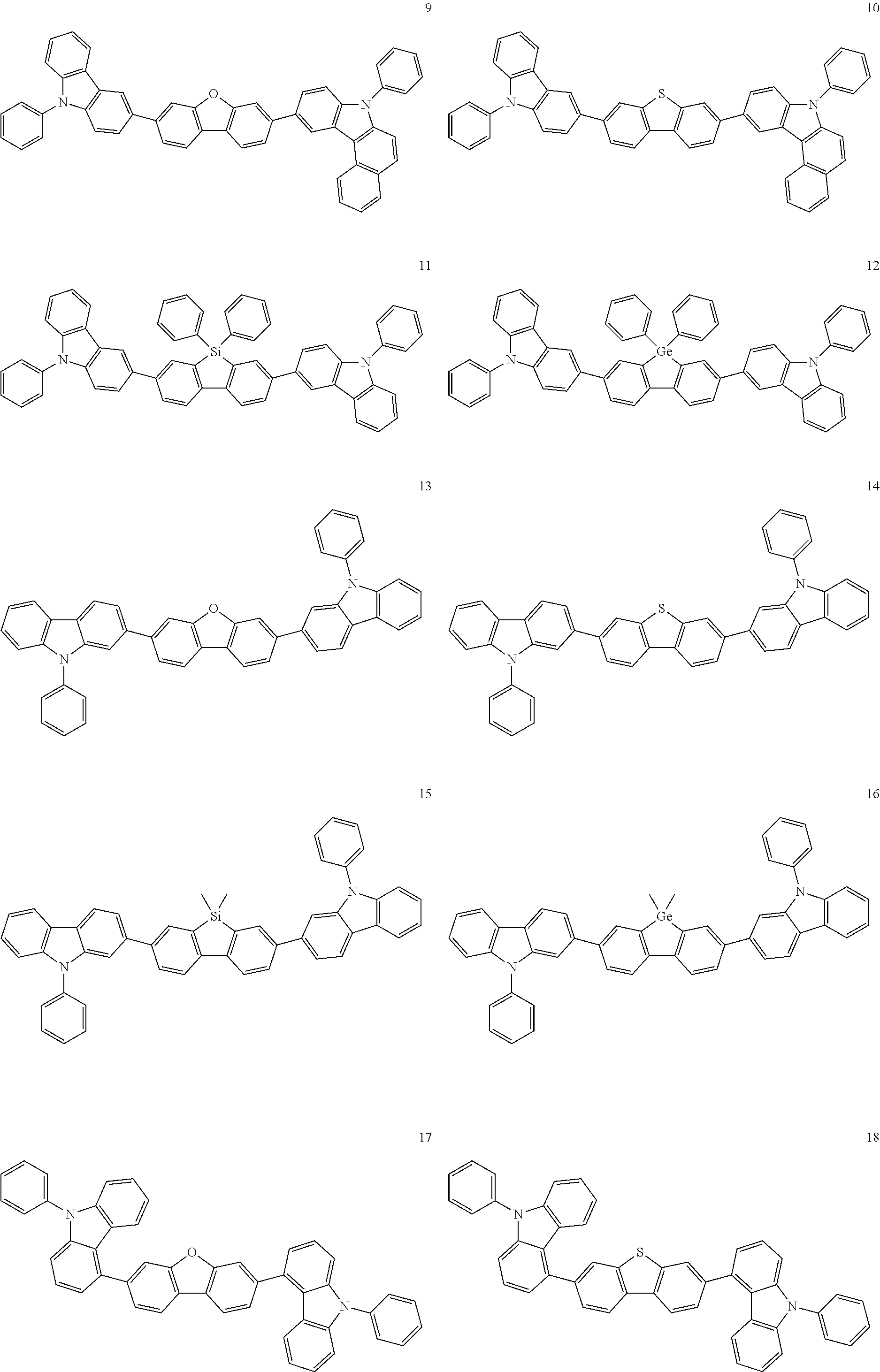

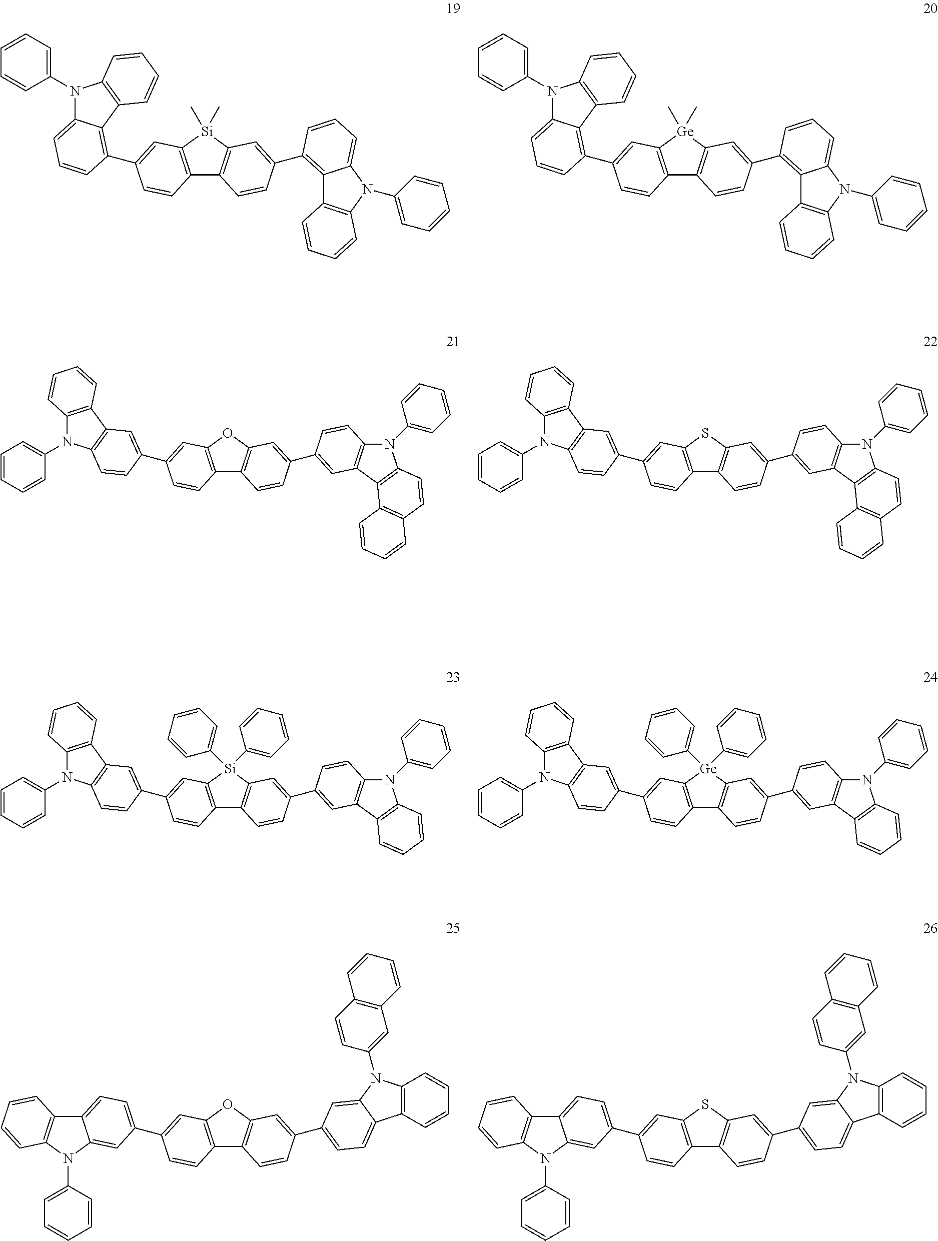

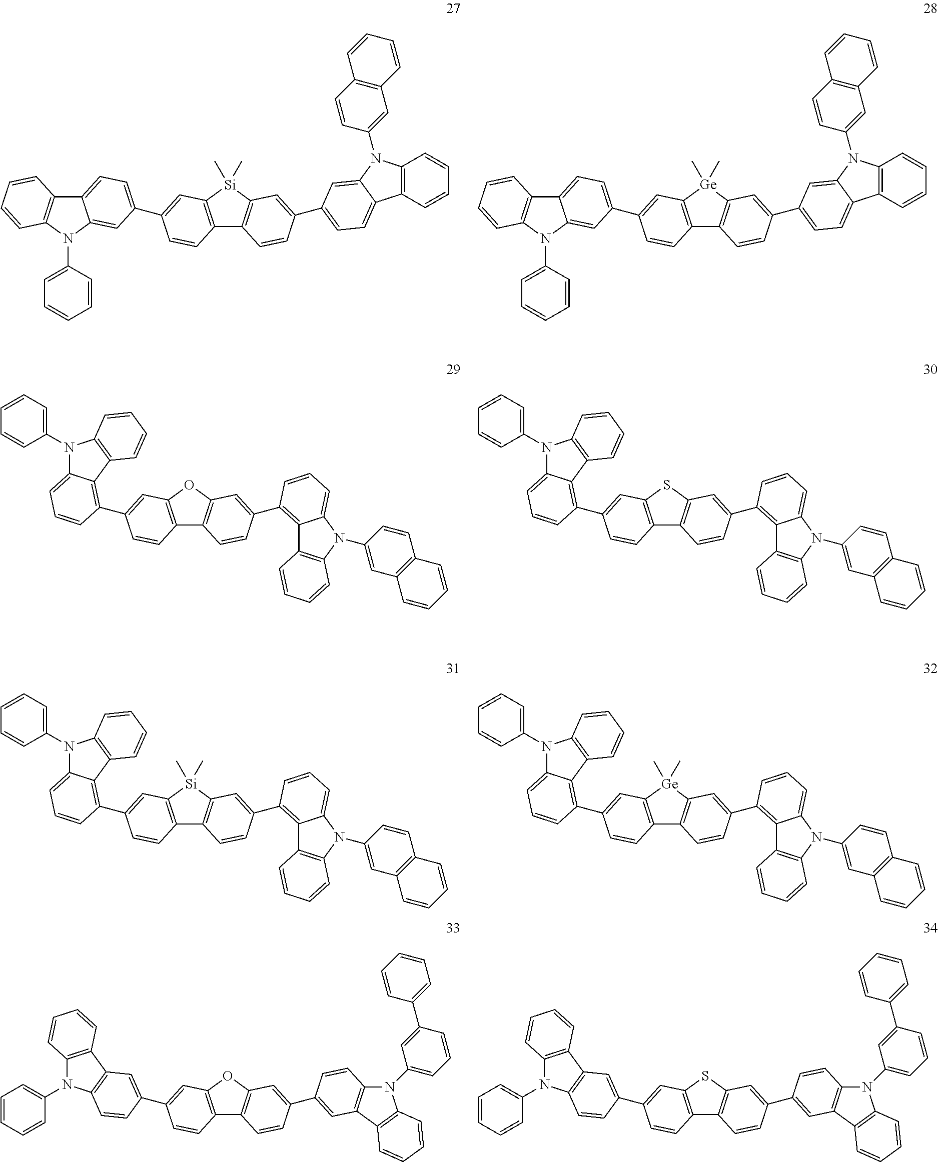

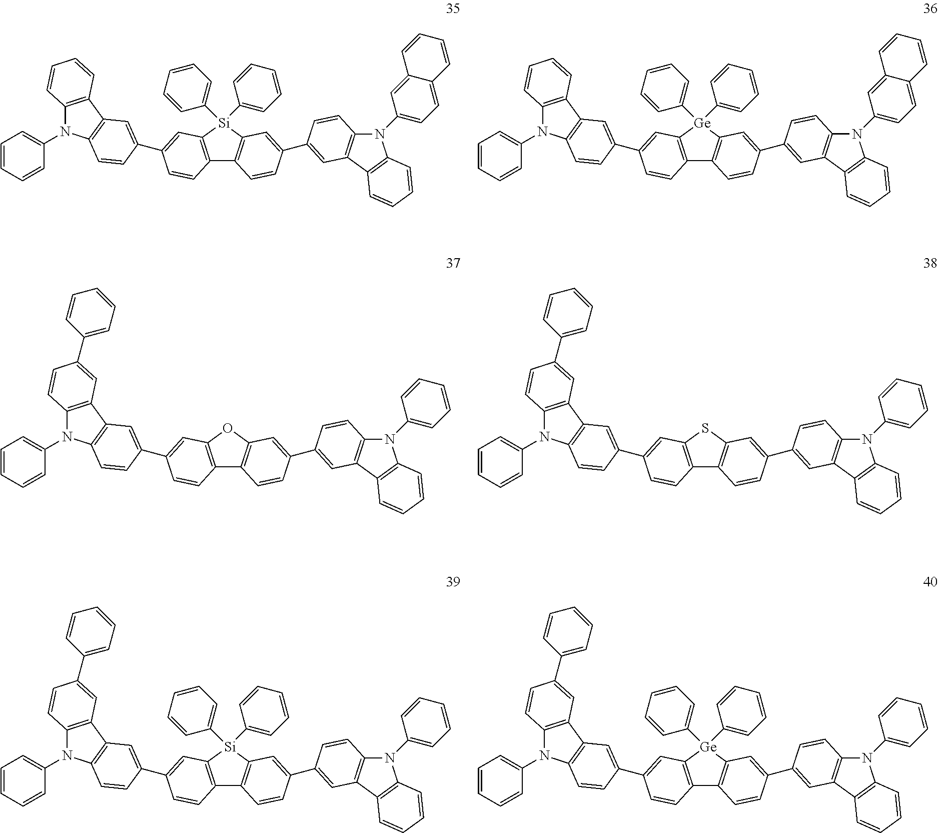

18. The organic EL device as claimed in claim 16, wherein the fused polycyclic aromatic compound is at least one of the following compounds a-1 to a-12: 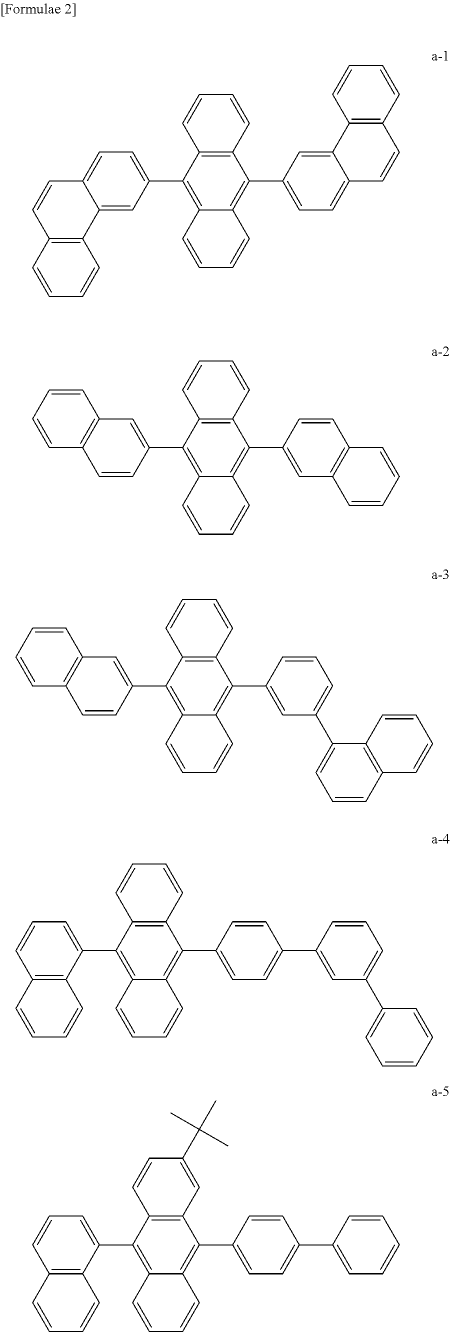 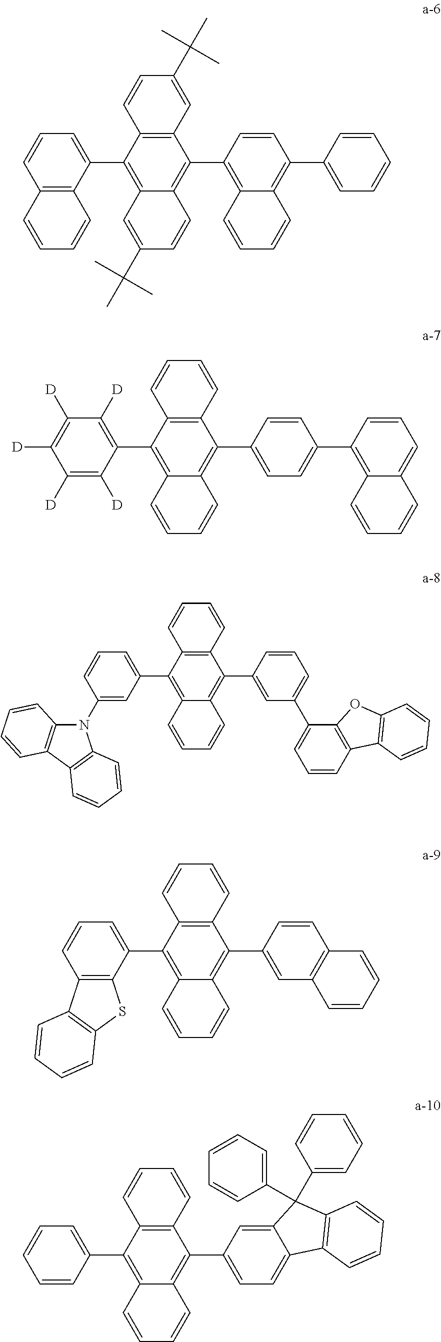 [chemical structure]
